(19) United States
(12) Patent Application Publication
(10) Pub. No.: US 2025/0031998 A1
(43) Pub. Date: Jan. 30, 2025

(54) WIRELESS BATTERYLESS SOFT SENSORS FOR AMBULATORY CARDIOVASCULAR HEALTH MONITORING

(71) Applicant: Georgia Tech Research Corporation, Atlanta, GA (US)

(72) Inventors: Woon-Hong Yeo, Atlanta, GA (US); Matthew Guess, Atlanta, GA (US)

(21) Appl. No.: 18/780,633

(22) Filed: Jul. 23, 2024

Related U.S. Application Data
(60) Provisional application No. 63/528,600, filed on Jul. 24, 2023.

(51) Int. Cl. A61B 5/11; A61B 5/00

(52) U.S. Cl. CPC A61B 5/1102; A61B 5/0002; A61B 5/6801; A61B 2562/0261; A61B 2562/164

(57) ABSTRACT

An exemplary embodiment of the present disclosure provides a cardiovascular sensing system, comprising a sensor, an external antenna, and a controller. The sensor can be configured to be worn by a user. The sensor can comprise an inductive capacitive (LC) circuit. The sensor can be configured to deform in response to a strain induced by vibrations of the heart of the user. The external antenna can be inductively coupled to the sensor. The external antenna can be configured to receive a signal from the LC circuit. The controller can be configured to determine a seismocardiogram (SCG) of the user based, at least in part, on the signal received by the LC circuit.

### CROSS-REFERENCE TO RELATED APPLICATIONS

This application claims the benefit of U.S. Provisional Application Ser. No. 63/528,600, filed on 24 Jul. 2023, which is incorporated herein by reference in its entirety as if fully set forth below.

### GOVERNMENT LICENSE RIGHTS

This invention was made with government support under Agreement No. GR00006395 awarded by National Science Foundation. The government has certain rights in the invention.

### FIELD OF THE DISCLOSURE

The various embodiments of the present disclosure relate generally to sensors.

### BACKGROUND

Cardiovascular diseases (CVD) account for over 19 million deaths annually in the United States. Early detection and diagnosis of this broad group of diseases are imperative in treatment. The clinical standard for detecting cardiovascular abnormalities is the electrocardiogram (ECG). However, ECG alone cannot capture a holistic view of heart function. Therefore, numerous other cardiovascular signals have offered crucial information to assist in the detection of CVD. One of these signals is the seismocardiogram (SCG). SCG is traditionally measured using an accelerometer to capture the local vibrations of the heart. While ECG measures myocardial conduction, SCG measures myocardial contractions and can capture the timing of fiducial points, such as mitral valve openings and closings and atrial valve openings and closings, which ECG cannot capture. SCG is typically measured using integrated circuit (IC) accelerometers. However, these accelerometer systems are rigid and bulky and, therefore, more uncomfortable to wear for long periods. In addition, accelerometers are also prone to the global inertia of the body. Although thin and flexible electronics are now becoming a widespread research field, exploring soft sensors for SCG is relatively unexplored. Soft and flexible sensors can offer solutions to these problems. A conventional system demonstrated a flexible serpentine interconnecting device over a rigid printed circuit board. However, the fabrication was still largely dependent on cleanroom processes and photolithography.

Studies have confirmed the effectiveness of piezoelectric strain sensors, fiber-optic strain sensors, and resistive strain sensors for SCG. All of these are improvements on the traditional MEMS accelerometers, yet they still require additional components for transmission. Adding the seamless wireless capability to these thin sensors is essential for ambulatory monitoring and patient comfort. Standard accelerometer-based SCG sensors have communicated data using Bluetooth, WiFi, and Zigbee. On the other hand, thin-film SCG sensors have only been measured wirelessly using active near-field communication (NFC) components. Additionally, these sensors are still limited by rigid circuit components and power consumption limits. All the current SCG sensors are either limited by low-throughput fabrication processes or bulky hardware for transmission. Thus, the embodiments disclosed herein aim to combine the comfort of thin film SCG sensors, a rapid, one-step fabrication method, and passive wireless capability.

### BRIEF SUMMARY

An exemplary embodiment of the present disclosure provides a cardiovascular sensing system, comprising an SCG sensor, and external antenna, and a controller. The SCG sensor can be configured to be worn by a user. The SCG sensor can comprise an inductive capacitive (LC) circuit. The SCG sensor can be configured to deform in response to a strain induced by vibrations of a heart of the user. The external antenna can be inductively coupled to the SCG sensor. The external antenna can be configured to receive a signal from the LC circuit. The controller can be configured to determine a SCG of the user, based at least in part, on the signal received by the LC circuit.

In any of the embodiments disclosed herein, the LC circuit can comprise a strain sensor and an inductive coil.

In any of the embodiments disclosed herein, the strain sensor can comprise a plurality of interdigitated electrodes.

In any of the embodiments disclosed herein, the plurality of interdigitated electrodes can be arranged in serpentine structures.

In any of the embodiments disclosed herein, the plurality of interdigitated electrodes can be configured to deform in response to a strain induced by vibrations of a heart of the user.

In any of the embodiments disclosed herein, the plurality of interdigitated electrodes can be configured such that deformation of the plurality of interdigitated electrodes alters a capacitance of the strain sensor.

In any of the embodiments disclosed herein, the SCG sensor can be encapsulated by an elastomeric material.

In any of the embodiments disclosed herein, the SCG sensor can be a passive sensor.

In any of the embodiments disclosed herein, the SCG sensor may not comprise an integrated circuit.

In any of the embodiments disclosed herein, the signal received by the antenna can correspond to the S₁₁ parameter.

In any of the embodiments disclosed herein, the S11 parameter can be measured at a stimulus frequency less than a resonant frequency of the sensor.

Another embodiment of the present disclosure provides a method of sensing a seismocardiogram (SCG) of a user, comprising: interrogating, with at least one wireless signal from an external antenna, a SCG sensor worn by a user, the SCG sensor comprising an inductive capacitive (LC) circuit, wherein the SCG sensor deforms in response to a strain induced by vibrations of a heart of the user; receiving, at the external antenna, a responsive signal from the LC circuit; and generating a SCG for the user based, at least in part, on the received responsive signal.

In any of the embodiments disclosed herein, interrogating the SCG sensor can comprise: interrogating the SCG sensor with a first wireless signal having a first frequency; and interrogating the SCG sensor with a second wireless signal having a second frequency different than the first frequency.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIGS. 1A-C provide an overview of an exemplary wireless soft sensor system for cardiovascular health monitoring, in which FIG. 1A provides a soft capacitive sensor mounted on the chest for wireless detection of data, FIG. 1B provides a schematic of an exemplary inductive coupling measurement method, and FIG. 1C illustrate measurements of seismocardiography, pulse, and heart rate, all in accordance with exemplary embodiments of the present disclosure.

FIG. 2A provides a schematic of the sensor layers in an exemplary device, FIG. 2B illustrates an exemplary sensor with a close-up of interdigitated electrodes arranged as serpentine structures, FIG. 2C illustrates the exemplary sensor mechanism with and without strain, FIG. 2D illustrates sensor capacitance as a function of strain, FIG. 2E illustrates capacitance changes of the sensor during 100 stretching cycles, FIG. 2F illustrates resonant frequency sweeps at different strains (the resonant frequency increases with increasing sensor strain, FIG. 2G illustrates resonant frequency at low strain values, and FIG. 2H illustrates resonant frequency at high strain values, all in accordance with exemplary embodiments of the present disclosure.

FIG. 3A provides a schematic of an exemplary inductive coupling method of detecting signals using the wireless soft sensor, FIG. 3B illustrates the S₁₁ signal at the resonant frequency at different distances between the coils (the coils are kept concentric), and FIG. 3C illustrates the quality factor from resonant frequency sweeps at different distances between the coils, all in accordance with exemplary embodiments of the present disclosure.

FIG. 4A illustrates the exemplary device on the lower chest, FIG. 4B provides raw SCG signal and SCG signal filtered between 4 and 24 Hz, FIG. 4C provides SCG signal recorded during a breath hold, FIG. 4D provides ensemble averaged SCG signal based on peaks (in which the mitral valve opening (MO), mitral valve closing (MC), isovolumic movement (IM), aortic valve opening (AO), and aortic valve closing (AC) fiducial points are identified, FIG. 4E provides simultaneous recording of SCG from the reported device, a commercial accelerometer, and ECG, all in accordance with exemplary embodiments of the present disclosure.

FIG. 5 provides a flow chart of an exemplary method of sensing a seismocardiogram of a user, in accordance with some exemplary embodiments of the present disclosure.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

Various embodiments of the present disclosure can provide cardiovascular sensing systems, which can be batteryless, wireless, chipless, and/or passive soft SCG sensors that can be measured using inductive coupling.

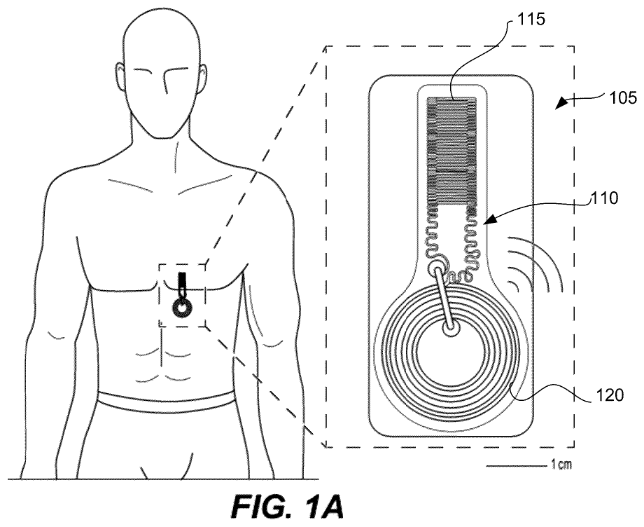

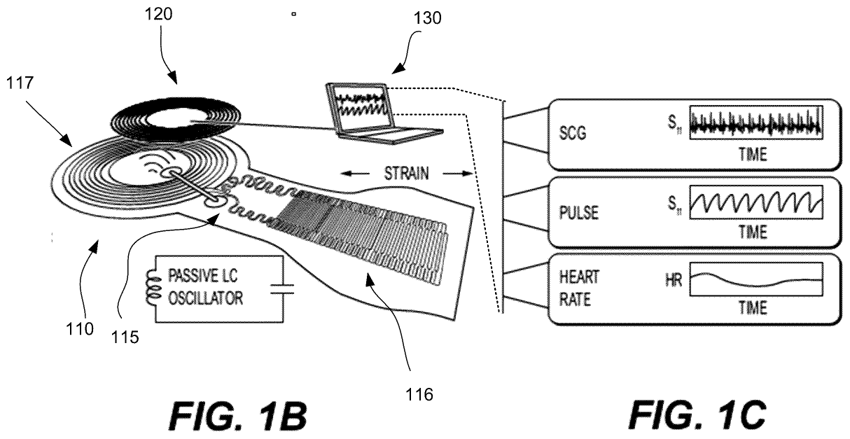

As shown in FIGS. 1A-B, an exemplary embodiment of the present disclosure provides a cardiovascular sensing system. The system can comprise an SCG sensor 110, and external antenna 120, and a controller 130. The SCG sensor can be configured to be worn by a user, e.g., on a chest area of the user proximate the user's heart. The SCG sensor can comprise an inductive capacitive (LC) circuit 115. The LC circuit 115 can comprise a strain sensor 116 coupled to an inductive coil 117. The SCG sensor 110, and in particular the strain sensor 116, can be configured to deform in response to a strain induced by vibrations of a heart of the user. The external antenna 120 can be inductively coupled to the SCG sensor 110 via the inductive coil 117.

Accordingly, the external antenna 120 can be configured to interrogate the inductive coil 117 with one or more wireless signals. In some embodiments, the one or more wireless signals comprise a plurality of signals having different stimulus frequencies. In some embodiments, the stimulus frequencies can be less than a resonant frequency of the sensor 110. In response to the wireless signals, the external antenna can receive one or more responsive signals from the LC circuit 115. The received signals can correspond to the S₁₁ parameter. The controller 130 can be configured to determine a SCG of the user, based at least in part, on the signal received by the LC circuit 115.

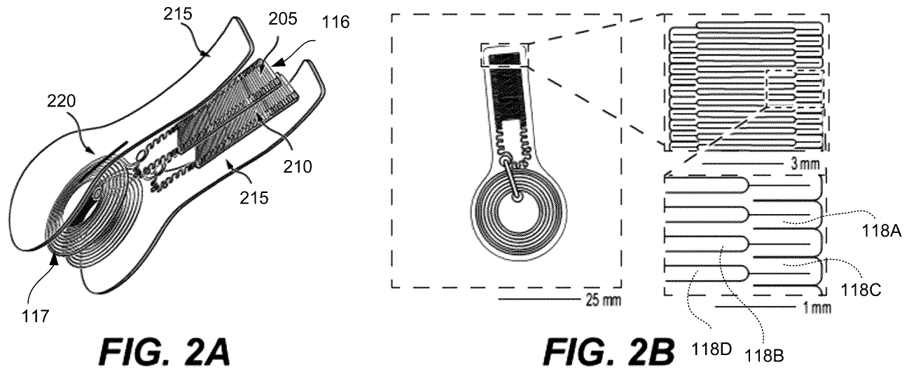

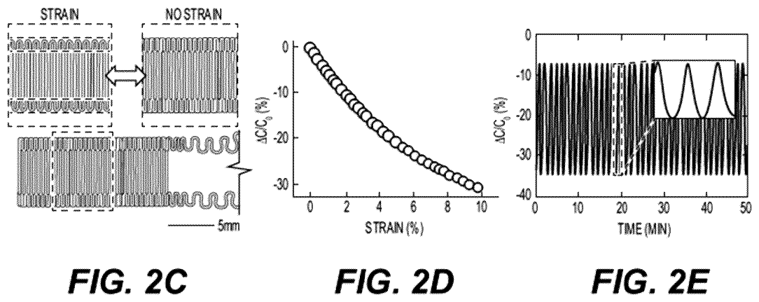

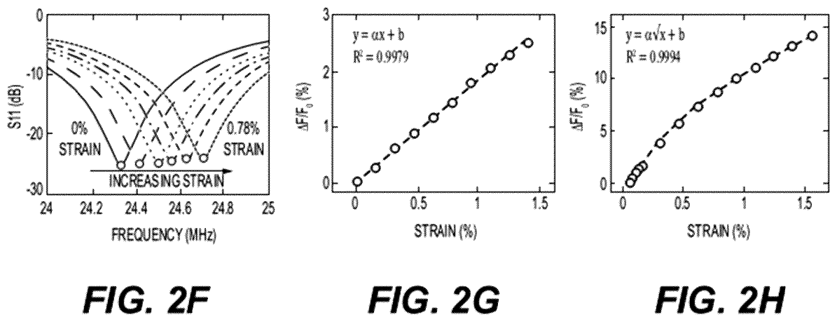

As shown in FIG. 2B, in some embodiments, the strain sensor 116 can comprise a plurality of electrodes 118A-D, which can be interdigitated. In some embodiments, the plurality of interdigitated electrodes 118A-D can be arranged in serpentine structures as shown in FIG. 2B. Accordingly, when the strain sensor experiences a strain (which can be induced by the beating of the user's heart), the strain sensor 116 can deform causing a spacing between the plurality of electrodes 118A-D to change, thus causing a change in capacitance of the strain sensor 116. This change in capacitance can then be measured by interrogating the inductive coil 117 with the external antenna 120. Based on the changes in capacitance over time, the controller 130 can determine certain characteristics of the user's heart, e.g., SCG.

In some embodiments, as shown in FIG. 2A, the strain sensor 116 and inductive coil 117 can be incorporated in a single layer of copper 205, which can be backed by a layer of polyimide 210. This can eliminate the need for cleanroom processes or other time-consuming methods during the manufacturing process. The thin sensor can be encapsulated with elastomer 215, allowing the device to attach to the user's skin with limited restrictions conformally. Furthermore, in some embodiments, the sensor 110 can be passive, thus not requiring any batteries or integrated circuit components, allowing the entire sensor to be flexible, stretchable, and nearly invisible to the user.

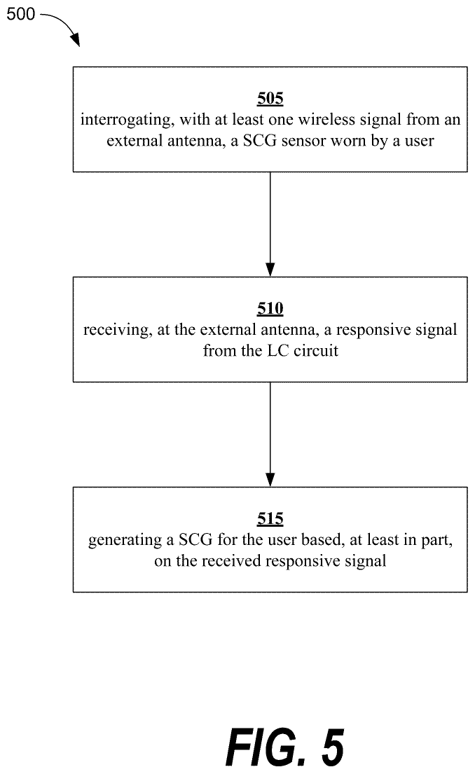

In addition to cardiovascular sensing systems, some embodiments of the present disclosure provide methods of sensing an SCG of a user. As shown in FIG. 5, an exemplary embodiments provides a method of sensing an SCG of a user 500. The method can comprise interrogating, with at least one wireless signal from an external antenna, a SCG sensor worn by a user 505. The method can further comprise receiving, at the external antenna, a responsive signal from the LC circuit of the SCG sensor 510. The method can further comprise generating a SCG for the user based, at least in part, on the received responsive signal 515. As discussed above, in some embodiments, interrogating the SCG sensor 505 can comprise interrogating the sensor with a plurality of signals, which can have different frequencies.

### EXAMPLES

The below examples are provided for illustrative and explanatory purposes only and should not construed as limiting the scope of the present disclosure.

### Results and Discussion

A wireless, ultrathin, soft sensor was developed with no rigid components like batteries and IC chips, demonstrating a seamless integration with the human skin. When mounted on the chest, this device can measure high-fidelity strain changes due to cardiac vibrations. FIG. 1A shows the mounting location of the wearable sensor when placed on the lower chest. The thin-film soft sensor can have a very small form factor and high mechanical compliance. This device, which can be encapsulated with elastomeric membranes, shows superior stretchability and flexibility with multi-modal bending, pulling, and twisting. FIGS. 1B-C show a schematic of the measurement system of the device, as well as its capabilities in passive wireless sensing using antennas. The exemplary device comprises a capacitive strain sensor 116 and an inductive coil 117, forming an LC oscillator circuit 115. This passive circuit offers wireless detection of the measured capacitive signals without rigid circuits and batteries. When mounted on the skin, this sensor can be inductively coupled with an external antenna 120 to record strain changes caused by cardiac vibrations. Overall, the soft sensor can measure SCG, pulse, and heart rate data. To validate the sensor, both an SCG measured with a commercial accelerometer and a single-lead ECG were used to compare the fiducial points. The fiducial points, including mitral valve closing (MC), isovolumic movement (IC), aortic valve opening (AO), aortic valve closing (AC), and mitral valve opening (MO), showed a strong association between the reported device and the commercial accelerometer.

FIG. 2A shows the layered structure of an exemplary developed chip-less, battery-less sensor. The entire device's thickness is 471 μm. The fabrication of the sensor can require only the patterning of one layer. Using a femtosecond laser, the sensor can be fabricated by micromachining a 6-μm copper foil 205 backed by polyimide (PI) 210. Laser micromachining was chosen as the fabrication process since it represents a promising alternative to traditional cleanroom lithography processes. To avoid direct contact with the human skin, the sensor can be encapsulated in a low-durometer silicone elastomer 215, providing conformal contact with the skin. It can also be helpful for the elastomer to be as thin as possible to maximize conformality to the skin. Since copper may not be stretchable, the fingers can be supported by connections with serpentine structures (as shown in FIG. 2B). The laser micromachining process can allow interdigitated electrode spacings to be as small as 8 μm, though the disclosure is not so limited. This can be important since the spacing between the interdigitated electrodes of the capacitive strain sensor can determine the sensitivity. As the sensor is stretched, these serpentine structures can expand to allow a large difference in spacing between the fingers. In addition, the serpentine pattern can change between the coil and the sensor to enable strain at the interface while minimizing stress on the copper (FIG. 2C). A bridge made of commercial high gauge enameled magnet wire 220 can be soldered across the pads connecting the interior of the coil 117 with the end of the capacitive sensor 116. To show the reliability of the exemplary sensor, it was stretched and unstretched for 100 cycles. FIG. 2D shows repeatable capacitance measurements with negligible hysteresis as a function of strain. The sensor also shows great stability and reliability; the capacitances stay constant for 100 cycles (FIG. 2E). To measure the relationship between the S₁₁ parameter and the strain, an experiment was conducted where continuous frequency sweeps were conducted at varying sensor strains. FIG. 2F shows a consistent resonant shift based on the strain. This was helpful for the device's design as the S₁₁ parameter can be the measurement used for capturing the SCG signal. The sensor shows an excellent linear resonant frequency response in low strains (FIG. 2G). This can be especially important for SCG measurement as the skin strain can be on the micrometers scale. The sensor showed repeatable measurements at each strain (n=4), indicating low hysteresis and high signal repeatability. Even at higher strains, the response is still predictable using a square root curve fit (FIG. 2H).

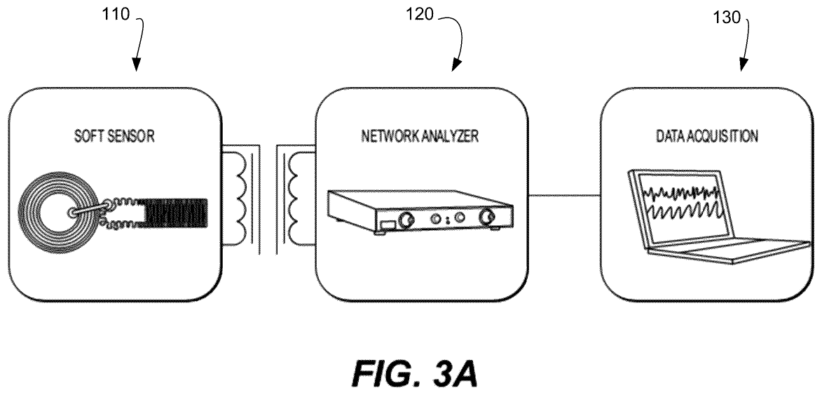

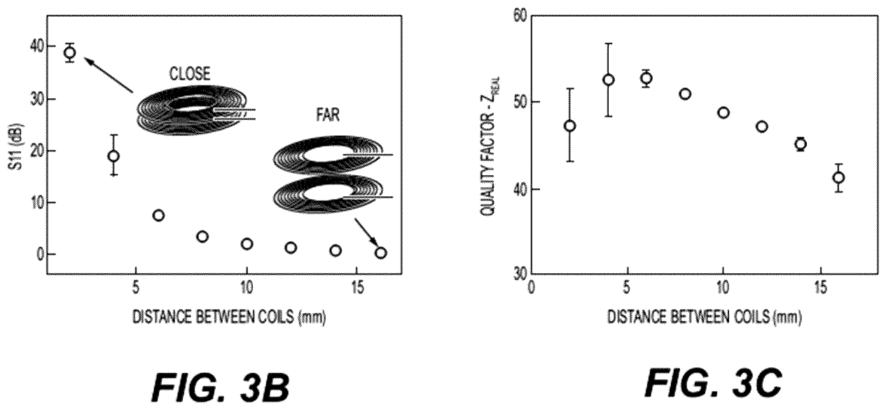

The resonance of the exemplary LC circuit can be measured using an external coil antenna 120 and vector network analyzer. FIG. 3A summarizes the inductive coupling method, system setup, and experimental results. FIG. 3A demonstrates how the sensor can be coupled with a vector network analyzer and a data acquisition system. As the capacitance of the sensor decreases, the resonance frequency can increase, as described in Note 1.

Note 1: The equation relating inductance and capacitance to resonant frequency is as follows:

$${F}_{R} = \frac{1}{\sqrt{LC}}$$

where F_{R} is resonant frequency, L is inductance, and C is capacitance.

To measure the SCG signal, the S₁₁ parameter can be measured at a stimulus frequency slightly lower than the resonance frequency. When the resonance frequency is increased, the S₁₁ of the selected stimulus frequency can be increased, which enables fast measurement of the SCG signal. FIG. 3B shows the S₁₁ value at the resonant frequency. As expected, the signal quality decreases as the distance between the coils increases. The coupling shows the highest quality factor at approximately 6 mm between the coils (FIG. 3C). The quality factor is calculated in Note 2.

Note 2: The equation used to calculate quality factor is as follows:

$$Q = \frac{{F}_{R}}{B}$$

where Q is quality factor, F_{R} is resonance frequency, and B is the −3 dB bandwidth, described as the range of frequencies for which the amplitude is at least half its peak value.

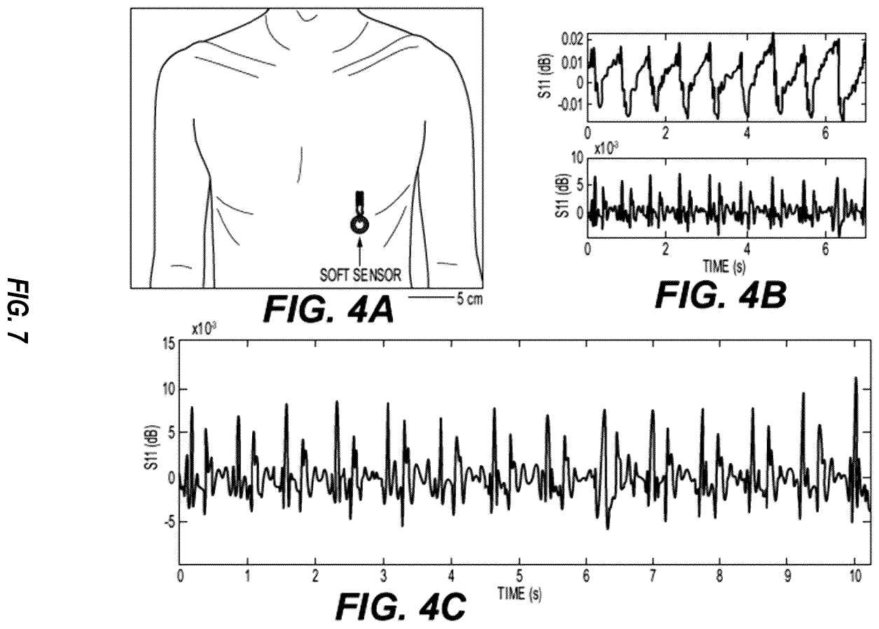

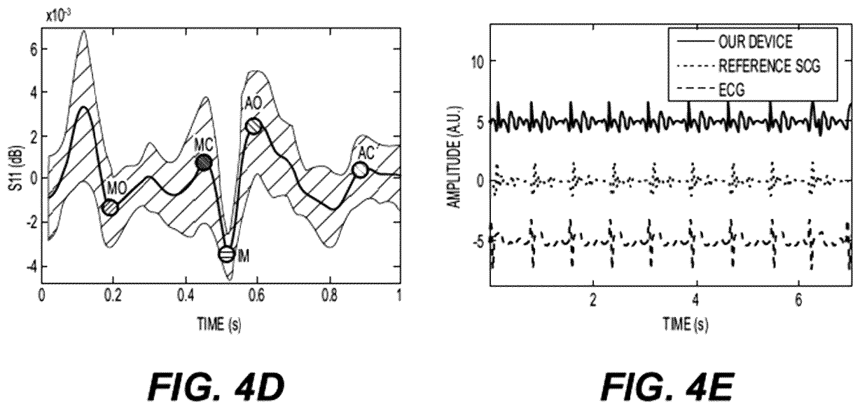

A set of human subject studies validated the exemplary device's performance in detecting SCG signals on the skin. FIGS. 4A-E summarizes the experimental results of high-quality detection of heart vibration data. A subject wears the soft sensor patch on the lower chest, as shown in FIG. 4A. The ultrathin sensor makes seamless integration and intimate contact with the skin without causing discomfort. In this study, it was found that the lower chest location (slightly below and to the right of the left nipple) can be the best place to receive large signal amplitudes by the sensor. This location also gives the advantage of visualizing the pulse rate, which can be seen in the raw data. But, the pulse data can be removed in the SCG signal detection by using digital filtering. FIG. 4B shows the measured raw SCG signals (top graph) and filtered signals (bottom graph). First, a 1^{st}-order low pass filter at 24 Hz is applied, and a 3^{rd}-order high pass filter at 4 Hz is used. These filters remove baseline wander and body movements in the signal analysis. In the graph of FIG. 4C, the SCG signals are clearly visible for 10 seconds. During this specific measurement, the subject was instructed to hold their breath to minimize motion artifacts. With the system setup, the soft sensor can measure meaningful cardiovascular signals, as demonstrated in FIG. 4D, showing the ensemble average using the beats of data as well as the MO, MC, IM, AO, and AC fiducial points. FIG. 4E shows the simultaneous signal measurement of an exemplary soft sensor and commercial SCG reference and ECG reference systems. The SCG signals show an excellent agreement on the locations of the S1 and S2 complexes. The soft device can also offer the ability to measure systolic time intervals from simultaneous ECG and SCG collection.

This disclosure demonstrates a wireless wearable soft capacitive sensor patch that can measure high-fidelity SCG signals on the skin without using batteries and rigid circuits. The entire device's thickness can be less than 1 mm, providing an imperceptible lamination to the skin without discomfort. This device is the first to wirelessly read SCG data accurately without wires or rigid chip components. The highly sensitive strain sensor in the patch can measure heart vibrations on the chest and identify fiducial points corresponding to cardiac events using inductive coupling. This stretchable thin-film sensor offers usefulness in ambulatory and remote healthcare applications where mechano-acoustic signals are essential, such as arrhythmia detection or obstructive sleep apnea. A sensor array may provide benefits by offering high amplitude signals near important heart locations, such as near the atrial and mitral valves. A dense sensor array can also help to mitigate motion artifacts by eliminating common noise elements in each sensor.

### EXPERIMENTAL

### Assembly of Passive Sensor

First, a glass slide was spin-coated with silicone elastomer (Ecoflex 00-30, Smooth-On, Inc.) at 500 rpm for 30 s and cured in an oven at 60° C. for 20 min. On a separate glass slide, polydimethylsiloxane (Sylgard 184, Sylgard) was spin-coated at 800 rpm for 60 s. Copper foil (6 μm, BR0214, MSE Supplies LLC) was then laid across the slide. PI (PI2545, HD MicroSystems) was spin-coated onto the copper foil at 800 rpm for 60 s and cured on a hot plate at 240° C. for 60 min. Afterwards, the copper and PI was transferred to the glass slide coated with elastomer. The sensor was then patterned on a laser micro-machining system (Femtosecond Laser Micro-Machining System, OPTEC). A bridge made of commercial high gauge enameled magnet wire was soldered across the pads connecting the interior of the coil with the end of the capacitive sensor. Finally, elastomer was spin-coated across the device at 500 rpm for 30 s and the device outline was cut out using laser micromachining. The assembly steps are shown in Supplementary FIG. 1.

### Wireless Transmission

The capacitive sensor and coil form an LC circuit with a varying capacitive value. When the sensor's capacitance decreases, the resonant frequency of the circuit increases. A vector network analyzer (Tektronix TTR506A) was connected to a spiral receiving antenna. A custom MATLAB program was used to record the S₁₁ parameter at a stimulus frequency close to the resonant frequency. The resonant frequency was determined by locating the frequency at the minimum Sn value in the range of 10 MHZ-60 MHz. The recording stimulus frequency was then determined by subtracting 0.1 MHz from the resonant frequency.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

What is claimed is:

1. A cardiovascular sensing system, comprising:
a seismocardiogram (SCG) sensor configured to be worn by a user, the SCG sensor comprising an inductive capacitive (LC) circuit, wherein the SCG sensor is configured to deform in response to a strain induced by vibrations of a heart of the user;
an external antenna inductively coupled to the SCG sensor, the external antenna configured to receive a signal from the LC circuit; and
a controller configured to determine a SCG of the user, based at least in part, on the signal received by the LC circuit.

2. The system of claim 1, wherein the LC circuit comprises a strain sensor and an inductive coil.

3. The system of claim 2, wherein the strain sensor comprises a plurality of interdigitated electrodes.

4. The system of claim 3, wherein the plurality of interdigitated electrodes are arranged in serpentine structures.

5. The system of claim 3, wherein the plurality of interdigitated electrodes are configured to deform in response to a strain induced by vibrations of a heart of the user.

6. The system of claim 5, wherein the plurality of interdigitated electrodes are configured such that deformation of the plurality of interdigitated electrodes alters a capacitance of the strain sensor.

7. The system of claim 1, wherein the SCG sensor is encapsulated by an elastomeric material.

8. The system of claim 1, wherein the SCG sensor is a passive sensor.

9. The system of claim 1, wherein the SCG sensor does not comprise an integrated circuit.

10. The system of claim 1, wherein the signal received by the antenna corresponds to the S₁₁ parameter.

11. The system of claim 1, wherein the S11 parameter is measured at a stimulus frequency less than a resonant frequency of the sensor.

12. A method of sensing a seismocardiogram (SCG) of a user, comprising:
interrogating, with at least one wireless signal from an external antenna, a SCG sensor worn by a user, the SCG sensor comprising an inductive capacitive (LC) circuit, wherein the SCG sensor deforms in response to a strain induced by vibrations of a heart of the user;
receiving, at the external antenna, a responsive signal from the LC circuit; and
generating a SCG for the user based, at least in part, on the received responsive signal.

13. The method of claim 11, wherein the LC circuit comprises a strain sensor and an inductive coil.

14. The method of claim 12, wherein the strain sensor comprises a plurality of interdigitated electrodes, and wherein the plurality of interdigitated electrodes are configured to deform in response to a strain induced by vibrations of the heart of the user.

15. The method of claim 13, wherein the plurality of interdigitated electrodes are arranged in serpentine structures.

16. The method of claim 13, wherein deformation of the plurality of interdigitated electrodes alters a capacitance of the strain sensor.

17. The method of claim 11, wherein interrogating the SCG sensor comprises:
interrogating the SCG sensor with a first wireless signal having a first frequency; and
interrogating the SCG sensor with a second wireless signal having a second frequency different than the first frequency.

18. The method of claim 11, wherein the SCG sensor is a passive sensor.

19. The method of claim 11, wherein the signal received by the antenna corresponds to the Sn parameter.

20. The method of claim 11, wherein the S11 parameter is measured at a stimulus frequency less than a resonant frequency of the sensor.
